(19) United States
(12) Patent Application Publication
(10) Pub. No.: US 2021/0027649 A1
(43) Pub. Date: Jan. 28, 2021

(54) SYSTEMS AND METHODS FOR PROVIDING BEHAVIORAL TRAINING FOR USER ENGAGEMENT WITH MEDICAL DEVICES

(71) Applicants: Board of Regents, The University of Texas System, Austin, TX (US); The University Of North Texas Health Science Center At Fort Worth, Fort Worth, TX (US)

(72) Inventors: Brandy M. Roane, Fort Worth, TX (US); Eileen D. M. Clements, Euless, TX (US)

(21) Appl. No.: 16/938,371

(22) Filed: Jul. 24, 2020

Related U.S. Application Data
(60) Provisional application No. 62/879,357, filed on Jul. 26, 2019.

(51) Int. Cl. G09B 19/00; A61M 16/08; A61M 16/06; A61M 16/00

(52) U.S. Cl. CPC G09B 19/00; A61M 16/0875; A61M 16/06; A61M 16/0057; A61M 2205/3592; A61M 2016/0027; A61M 2016/003; A61M 2205/3368; A61M 16/0051

(57) ABSTRACT

Various examples of a system and method for providing behavioral training for user engagement with medical devices are described. In one example, a behavioral training system is configured for use with a positive airway pressure (PAP) system that provides PAP therapy to a user. The behavioral training system can include one or more sensors configured to capture measured data and configured to measure parameters pertaining to engagement of a user with a medical device. The system also includes a user interface configured to receive user input data from the user. The system also includes program instructions to analyze measured data and user input data using an expert system, determine, in real time, whether an event related to engagement of the user with the medical device has occurred, and in response to the event, initiate a behavioral training protocol based on an event status.

### CROSS-REFERENCE TO RELATED APPLICATION

This application claims the benefit of and priority to U.S. Provisional Patent Application No. 62/879,357, titled “SYSTEMS AND METHODS FOR PROVIDING BEHAVIORAL TRAINING IN RELATION TO POSITIVE AIRWAY PRESSURE THERAPY,” filed on Jul. 26, 2019, the entire contents of which is hereby incorporated herein by reference.

### BACKGROUND

Obstructive sleep apnea (OSA) is a sleep-related breathing disorders in which a person has pauses in breathing while sleeping because of a blockage of airflow. Each pause can last for a few seconds to a few minutes and may happen many times a night. As the disorder disrupts normal sleep, those affected may experience sleepiness or feel tired during the day, and may be vulnerable to a variety of long-term adverse consequences, such as increased risk of mortality, hypertension, heart attack, stroke, obesity, diabetes, depressed mood, and anxiety.

The common treatment for OSA is positive airway pressure (PAP) therapy in which pressurized air is delivered into the airway of the individual by a PAP machine that generates pressurized air that travels through a hose from the machine to a mask worn on the face. While this treatment can be highly effective, adherence rates to PAP therapy are alarmingly low at 30 to 50% among the over 63 million U.S. adults with OSA. This is unfortunate as, just like individuals with hypertension and other chronic conditions, persons with OSA need to follow their prescribed treatment regimen in order to control their disease and mitigate adverse health outcomes.

As PAP therapy is a life-long treatment, adherence to the therapy requires multifactor interventions that incorporate behavioral training with the prescribed therapy in order to address the aspects of behavior change required for the individual to use the PAP machine as prescribed. Unfortunately, PAP machines are not currently configured to provide such behavioral training. Accordingly, it can be appreciated that there is a need for a means to provide such training.

### BRIEF DESCRIPTION OF DRAWINGS

Many aspects of the present disclosure can be better understood with reference to the following drawings. The components in the drawings are not necessarily drawn to scale, with emphasis instead being placed upon clearly illustrating the principles of the disclosure. In the drawings, like reference numerals designate corresponding parts throughout the several views.

FIG. 1 illustrates a schematic diagram of an example of a system for providing behavioral training, a behavioral training system (BTS), used in conjunction with a positive airway pressure (PAP) system according to various embodiments described herein.

FIG. 2 illustrates a flow diagram of an example of the operation of the BTS shown in FIG. 1 according to various embodiments described herein.

FIG. 3 is a flow diagram of an example of a “mask removed” behavior training protocol to facilitate a change in user engagement and/or adherence with the PAP device using the BTS shown in FIG. 1 according to various embodiments described herein.

FIG. 4 is a flow diagram of an example of a “no or low use” behavior training protocol to facilitate a change in user engagement and/or adherence with the PAP device using the BTS shown in FIG. 1 according to various embodiments described herein.

FIG. 5 illustrates a comparison of data from the PAP machine, the developed monitoring system, and the manually introduced events when varying levels of leak were manually introduced at the mask according to various embodiments described herein.

### DETAILED DESCRIPTION

As described above, there is a need for a means to provide behavioral training to individuals with obstructive sleep apnea (OSA) to address the aspects of behavior change required for the individual to use a positive airway pressure (PAP) system long term. Disclosed herein are systems and methods for providing such behavioral training. In some embodiments, various data is collected about the PAP therapy using one or more sensors located external to the PAP system, and various information is collected from the PAP user using a user interface. This data and information are then used to understand the engagement of the user with their PAP therapy and evaluate the user's adherence to his or her PAP therapy. Based upon that evaluation, one or more behavioral training protocols can be executed that are specifically designed to address the behavioral aspects of use of the PAP system that may be interfering with the user adhering to the therapy.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure. Although OSA is used as an example of a breathing disorder, it should be appreciated that the systems and methods for providing behavioral training for user engagement with medical devices can be relied on for patients with central apnea, and other chronic or acute breathing conditions who are prescribed PAP therapy using a PAP system. Additionally, the systems and methods for providing behavioral training can be relied on for use with medical devices for treating a variety of chronic health conditions (e.g., positive airway pressure machines for obstructive sleep apnea, nebulizers for asthma, glucose monitors for diabetes) to convey meaningful information to the user of the medical device regarding how the user is engaging with the medical device in real-time. Similarly, the systems and methods for providing behavioral training can be relied on for use with medical devices for acute conditions, such as breast pumps for breastmilk expression.

In the context of engagement of a user with a prescribed medical device, adherence to PAP therapy is predicted by a) increased interactions between the healthcare team and the patient, who is the user of the medical device, b) increased frequency of PAP therapy use from the start of treatment, and c) when the user notices improvement in overall functioning the next day after using PAP therapy. However, studies have found that only addressing these primary factors that predict adherence to PAP therapy does not yield improved long-term adherence to PAP therapy. When providers and staff increase contact with patients after they start PAP therapy via phone calls or other means, patients initially show higher use of PAP therapy. However, this increase in adherence quickly declines when these interactions involve the user receiving standard (non-behavioral) training, such as having a mask changed out or the PAP therapy pressure setting changed, or when additional education on general PAP therapy use is received. Similarly, when users are given web-based access to their PAP therapy data so they can self-monitor their use, PAP adherence initially increases. And, just like with increased patient contact offering non-behavioral training, a sharp decline is then observed in adherence when PAP therapy users do not receive any feedback with an interpretation of the data or other training.

Targeting the predictors of PAP therapy adherence does not produce the desired adherence because successful adherence to PAP therapy requires users to change their behavior. However, behavior change is difficult and usually unsuccessful without the right tools. Behavior change occurs best with targeted, specific feedback as the health behavior is happening. The most effective methods for improving adherence to PAP therapy use behavioral interventions or training. Behavioral training targets what fundamentally needs to change for patients to be adherent to the treatment, i.e., their behavior. Behavioral sleep medicine specialists are trained clinicians who assist patients with adherence by implementing empirically-validated behavioral training protocols that target the specific areas PAP users are struggling with (e.g., mask phobia, integrating PAP therapy into their lifestyle, residual insomnia). Unfortunately, only a limited number of clinicians have the training to provide these services (less than 1 certified behavioral sleep medicine specialist per 100 accredited sleep facilities). Consequently, an unmet need has arisen from the steadily increasing demand for behavioral training services that few providers are trained to deliver. Alternative mechanisms to deliver these services can address this unmet need.

Some PAP device manufacturers have developed apps around the three primary predictors of adherence, and preliminary data show improved PAP therapy adherence with use of an app; however, as noted above, prior research has shown that addressing these factors usually produces short-term improvements in adherence, but very little improvements long-term. This initial bump in adherence is driven by increased self-monitoring of the health behavior. Unfortunately, sustained monitoring does not promote improved PAP adherence, only lowers risk of discontinuation of PAP therapy. While the current technologies are not delivering increased adherence rates, moving the access to delivery of behavioral training into the home to tackle adherence could be a game-changer if patients are provided with the right tools.

In the context PAP therapy devices and prescribed use by patients, various examples of the systems and methods for providing behavioral training for user engagement with medical devices are described herein. The systems and methods for providing behavioral training are provided to monitor a patient's engagement with their prescribed PAP therapy device and deliver the necessary behavioral training in the home in order to manage and increase adherence, which is a necessary component in treating OSA. This type of technology can provide a behavioral intervention tailored to the user and in real-time.

This technology can be expanded beyond improving adherence to only PAP therapy. Bringing virtual behavioral health specialists into the home could be applied across multiple chronic conditions that require the use of a medical device, such as asthma and nebulizers or diabetes and blood glucose monitors. Virtual behavioral health specialists in the home could also be applied for acute conditions, such as breastmilk expression and breast pumps.

Described herein is an example of a behavioral training system (BTS) that is configured to monitor a user's engagement with their prescribed PAP machine and deliver the necessary behavioral training interventions in the home in order to manage and increase adherence of the PAP therapy. In one embodiment, the systems and methods for providing behavioral training for user engagement with medical devices includes: a sensor module that captures data on user engagement with their PAP device, an electronics unit that collects sensor data and transmits said data, a controller that processes data from sensors and from the user, manages the training module, and communicates information to the user, a training module that interprets processed data and initiates, terminates, and manages behavioral training protocols, memory that stores all user engagement data, user preferences, and behavioral training protocols, one or more behavior training protocols that determines information to collect from and/or send to the user to facilitate a change in engagement and/or adherence with their PAP device, and a user interface that allows for data to be input by the user and for information to be delivered to the user.

In an example, there can be one or more sensor modules. The one or more of the sensor modules can contain one or more air pressure sensors, audio sensors, inertial motion sensors, air flow sensors, light sensors, and/or temperature sensors. In another aspect, one or more of the sensor modules can be connected to one or more of the PAP device, PAP hose, and PAP mask. In another aspect, one or more sensor module can be used without connecting to any of the PAP device, PAP hose, and PAP mask. In another aspect, one or more sensor modules can be worn by the user.

In another example, the systems and methods for providing behavioral training for user engagement with medical devices can include wireless communication between one or more of the sensor modules, the electronics unit, controller, and user interface. In an aspect, the user interface, electronics unit, and the controller can be in a computing environment. In an aspect, the computing environment can include a computing device comprising at least one hardware processor. In an aspect, the user interface, electronics unit, and the controller can be housed together. In an aspect, the user interface can include a display device to display information, images, and/or rendered data. In an aspect, the user interface can include means to receive data or information from a user, such as buttons, a keyboard, touch screen, and the like. In another aspect, the user interface can be incorporated into a smart phone, tablet, or wearable device (such as a smart watch).

Descriptions of additional aspects are provided within the details of the examples discussed below. For example, information sent to the user, events that can be identified, details about the training modules and behavior protocols, and means of communication can be varied.

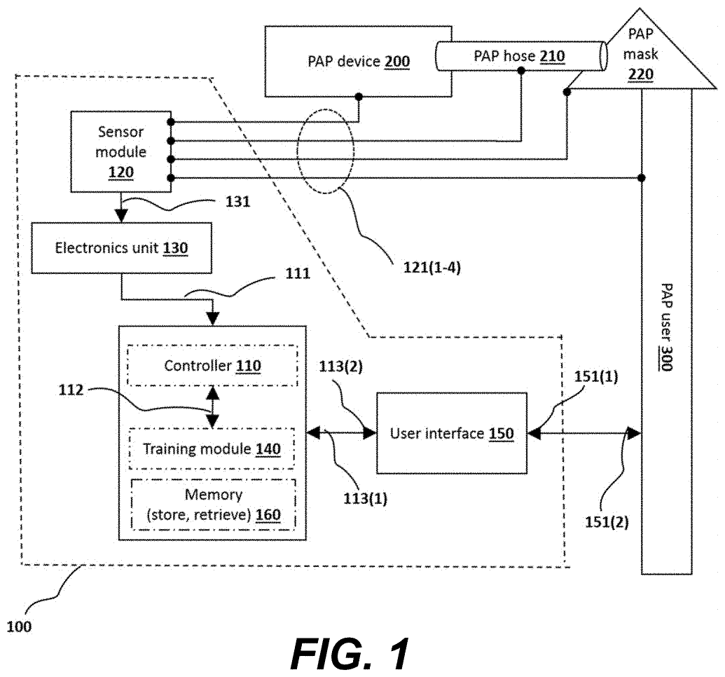

Illustrated in FIG. 1 is an example embodiment for the BTS 100, which can be used in association with a PAP system. The PAP system comprises a PAP device 200 that generates the pressurized air and a PAP hose 210 that delivers the pressurized air to a PAP mask 220, which is worn by a PAP user 300. As shown in the figure, the BTS 100 comprises a sensor module 120, an electronics unit 130, a computing device that comprises a controller 110, a training module 140, and memory 160, and a user interface 150. In some embodiments, some or all of these components can be housed together in a single integrated device. In addition, in some embodiments, the user interface 150 can be incorporated into a user device, such as smart phone, tablet, or wearable device (such as a smart watch).

The sensor module 120 includes one or more sensors that are associated with the components of the PAP system. In the illustrated embodiment, one or more sensors are associated with each of the PAP device 200, the PAP hose 210, and the PAP mask 220. In addition, one or more sensors can be associated with the PAP user 300. The various sensors collect data about the PAP system and user that relate to how the user is engaging with the PAP system. By way of example, the sensors can include one or more air pressure sensors, air flow sensors, audio sensors, inertial motion sensors, light sensors, and temperature sensors. The sensors are external to the PAP system itself, and do not require connection to the electronics or sensors that are inherently part of the PAP system.

The sensor data collected by the sensors is sent (131) to the electronics unit 130 for signal processing. Such processing can comprise, for example, amplification or digitization of the data. The electronics unit 130 sends (111) the processed data to the controller 110 of the computing device. The user interface 150 allows for data to be input by the user 300 (151-1) and for information to be delivered to the user (151-2). The controller 110 then combines and processes data from the electronics unit 130 and information from the user 300 through the user interface 150, 113-1. The controller 110 also manages 112 the training module 140 and communicates information to the user (113-2, 151-2) through the user interface 150. Notably, communication between the sensor module 120 and its various sensors, the electronics unit 130, the controller 110, memory 160, and the user interface 150 can be via a wired connection or a wireless connection.

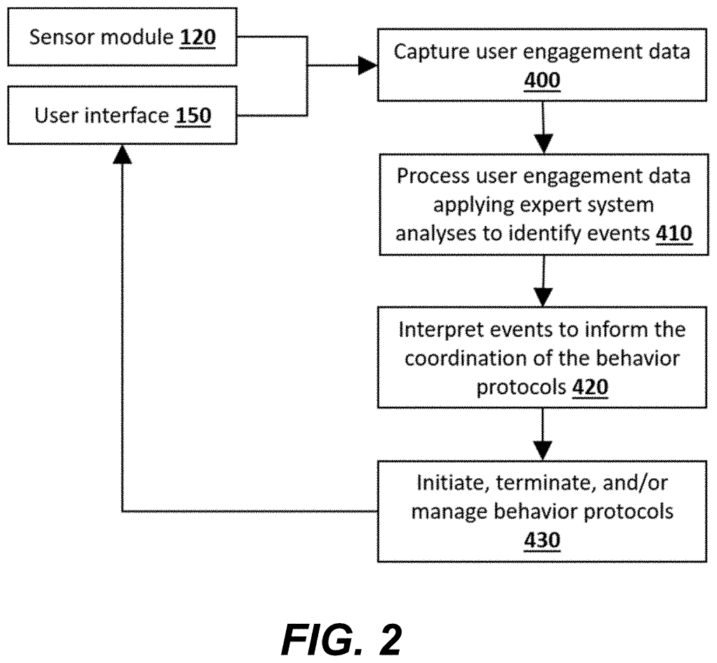

The training module 140 comprises software that uses expert system analysis to interpret the processed data 112 from the controller 110 to determine in real time occurrence of events related to the user's 300 engagement with the PAP system and initiates, manages, and terminates predetermined behavior protocols that are designed to modify the behavior of the user in a manner that will maintain or improve adherence to the PAP therapy regimen. In particular, the behavior protocols determine information to collect from the user (151-1) and/or to send to the user (151-2) to facilitate changes in the user's engagement and/or adherence with their PAP system. Examples of such behavior protocols and their execution are described in relation to FIGS. 2-4. Examples of information sent to the user (151-2) include tailored feedback, notifications, status information, instructions, questions, and alarms relating to the user's engagement with their PAP system.

The processed data from the electronics unit 130, information received from the user 151-1, 113-1, information sent to the user 113-2, 151-2, and information pertaining to behavior protocols are stored in and available to be retrieved from the memory 160. Events identified by the training module 140 related to user engagement may include sensed conditions such as “PAP machine on” (i.e., the PAP machine is running), “PAP machine off” (i.e., the PAP machine is not running), “Mask on” (i.e., the user has the mask on while PAP machine is on), “Mask off′ (i.e., the user has removed the mask while PAP machine is on”), “no or low use” (i.e., PAP therapy has not been used at night or has been prematurely terminated at night), and “Daytime practice session” (i.e., the user has engaged in a daytime practice session). The system and method for providing behavioral training for user engagement with medical devices is not limited to the above listed events provided as an example. For example, the events can be defined in the training module according to type of engagement of the user with the medical device, function of the medical device, and/or time elapsed between uses of the medical device. Further, the events are not limited to any particular type of medical device, but can be defined to convey meaningful information to the user of the medical device regarding how the user is engaging with the medical device in real-time.

The BTS applies its expert system analysis to contextualize these events based on circumstances that may include one or more of preceding events that have occurred, time of day, user preferences, and behavior protocol requirements. For instance, the occurrence of a “Mask off” event during the hours the PAP user 300 previously indicated as the sleep period taken in the context of a preceding “PAP machine on” event, would indicate that PAP therapy had been interrupted during sleep as a result of the mask being removed. This interpretation would signal the “Mask removed” protocol to be initiated.

Behavior protocols utilize communications with the user 300 such that the user can be prompted to provide information or take specific action that will adjust their behavior and thus impact their engagement with and adherence to their PAP therapy. As the user 300 does or does not provide information and/or take action, the training module 140 will continue to interpret processed data (112) received from the controller 110 in real-time, thereby allowing the training module to determine the occurrence of or change in events related to the user's engagement with their PAP device 200. This continuous process allows the training module 140 to initiate, manage, and terminate behavior protocols to cause a preferred change in the user's engagement with their PAP system, thus managing their adherence to their prescribed PAP therapy.

FIG. 2 illustrates an example of how the BTS 100 operates as a continual process to coordinate behavior protocols, wherein coordination includes the initiation and termination of individual behavior protocols as well as the management of multiple behavior protocols active at the same time. In this process, “user engagement data” are continually captured (block 400) and analyzed to identify events (block 410). Identified events are interpreted (block 420) to inform the coordination of the behavior protocols (block 430).

The “user engagement data” is comprised of data from the sensor module 120 and information from the user via the user interface 150. It is possible for more than one behavior protocol to be active at any time based on the identification and interpretation of events as they occur. When a behavior protocol is initiated, information in the form of questions, alarms, notifications, and/or instructions, etc. can be sent to the user through the user interface 150. Notably, the entire process illustrated in FIG. 2 for identifying and interpreting user engagement data occurs within the BTS 100 and does not involve receiving data communicated from any other system (such as the medical device or the PAP system) or a person other than the user, nor does it involve receiving any additional data besides what is already being collected through the sensor module 120 and the user interface 150.

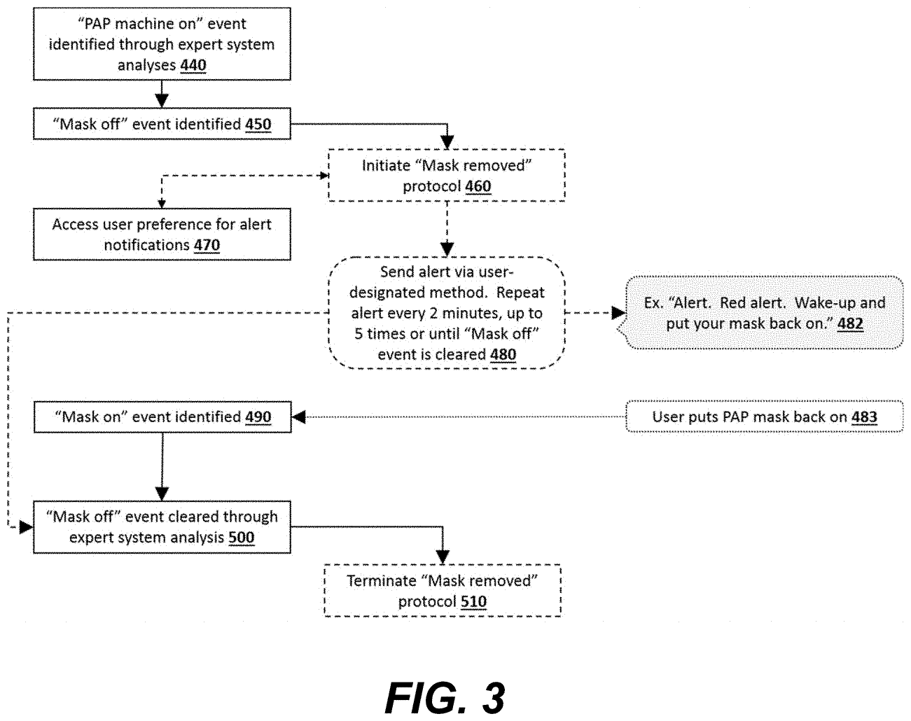

FIG. 3 provides an example of execution of a behavior protocol. More particularly, FIG. 3 shows an example of a behavior protocol called “Mask removed” being executed by the BTS 100. This protocol is initiated if it is determined that a user 300 has taken their PAP mask 220 off after having started to use their PAP system.

The first step in the process is the identification of the “PAP machine on” event through the aforementioned expert system analysis (block 440). The next step in the process is the identification of the “Mask off” event (block 450). Once both events have been identified in that order, and if no other events are identified, the next step in the process initiates the “Mask removed” behavior protocol 460. Once the “Mask removed” behavior protocol is initiated, an alert is sent to the user through a predetermined method (block 480). The predetermined method, along with additional user preferences, would have been previously requested by the BTS 100 and input by the user 300 through the user interface 150 and stored in memory 160. The behavior protocol accesses this information (block 470) in memory and provides alerts and notifications as needed. In this behavior protocol example, the alert stating “Alert. Red alert. Wake-up and put your mask back on.” (block 482) is sent to the user every 2 minutes. The alert is sent up to 5 times or until the “Mask off” event is cleared. The BTS 100 is continuously monitoring for new or changed events. While the “Mask off” behavior protocol is active, if the user puts their PAP mask 220 back on (block 483), the “Mask on” event is identified (block 490) causing the “Mask off” event to be cleared through the expert system analysis (block 500). Once the “Mask off” event is cleared, the behavior protocol terminates (block 510).

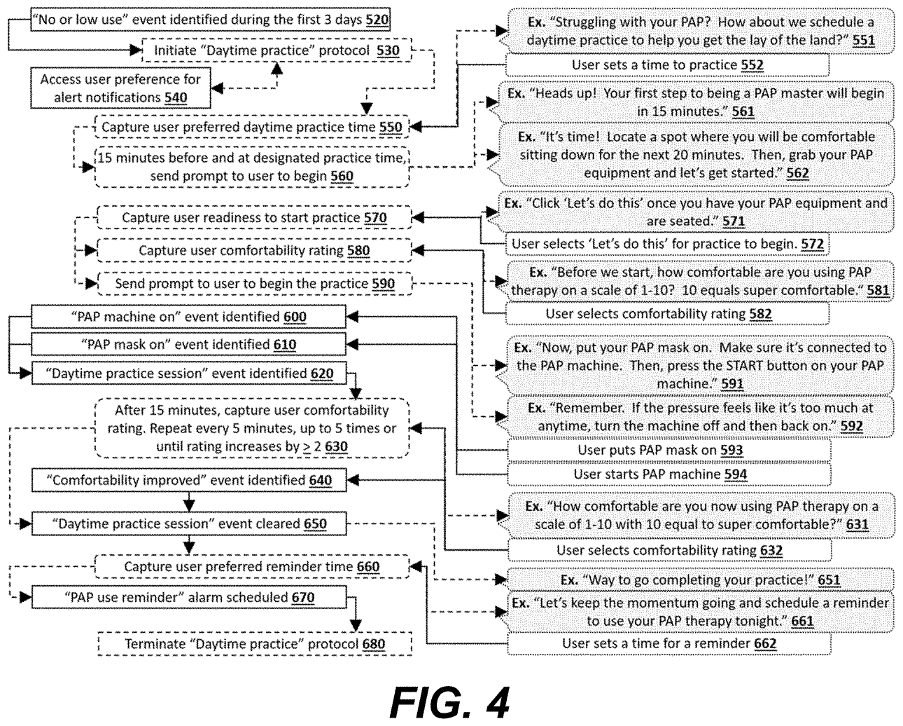

FIG. 4 shows a further example of a behavior protocol initiated by the BTS 100. In this case, the protocol is executed if it is determined that a user 300 has had “no or low use” of their PAP device 200 during the first 3 days from when the BTS 100 was first operated. This behavior protocol does not require the PAP device 200 to be in operation. Instead, this protocol is implemented for user engagement that indicates a user's lack of engagement with their PAP device 200. Once such an event is identified, the “Daytime practice” protocol is initiated (block 530). This protocol retrieves and stores user preferences for alert notifications and other information (block 540), as previously described in FIG. 3.

The next step of the behavior protocol is to “capture user preferred daytime practice time” (block 550), at which point an inquiry is sent to the user to capture said time. An example inquiry is “Struggling with your PAP? How about we schedule a daytime practice to help you get the lay of the land?” (block 551), which is followed by the user setting the preferred time for the daytime practice session to start (block 552).

The next step in this behavior protocol is to send a prompt to the user 15 minutes before and at the designated practice time to begin the practice session (block 560). Example prompts are: “Heads up! Your first step to being a PAP master will begin in 15 minutes.” (block 561) and “It's time! Locate a spot where you will be comfortable sitting down for the next 20 minutes. Then, grab your PAP equipment and let's get started.” (block 562).

The next step in this behavior protocol is to determine if the user is ready to begin the daytime practice (block 570). An example prompt is “Click ‘Let's do this’ once you have your PAP equipment and are seated.” (block 571), which is followed by the user indicating that they are ready to begin (block 572).

The next step in this behavior protocol is to capture the user's comfort rating to establish a baseline rating that will be used to determine the success of the practice session (block 580). An example prompt is “Before we start, how comfortable are you using PAP therapy on a scale of 1-10? 10 equals super comfortable.” (block 581), which is followed by the user selecting their comfortability rating (block 582).

The next step in this behavior protocol is to prompt the user to start the practice session (block 590). An example prompt is “Now, put your PAP mask on. Make sure it's connected to the PAP machine. Then, press the START button on your PAP machine.” (block 591). An example follow-up prompt with a helpful tip is “Remember, if the pressure feels like it's too much at any time, turn the machine off and then back on.” (block 592). Up to this step, the PAP device 200 does not need to be on in order for the BTS 100 to be operational and run a behavior protocol.

The next steps in this protocol are identification of “PAP machine on” and “PAP mask on” events (blocks 600, 610) when the user starts the PAP machine (block 594) and has the PAP mask on (block 593). These two events (“PAP mask on” and “PAP machine on”) during the “Daytime practice” protocol signal the system to identify the event “Daytime practice session” (block 620).

The next step in this behavior protocol is a loop that asks for and captures the user's comfort rating that begins after the designated practice session duration (block 630). In this example, the step begins 15 minutes after starting the practice session (block 620), and then continues every 5 minutes, up to 5 times or until the comfort rating increases by a value greater than or equal to 2 (block 630). At each of the time intervals, an example prompt to the user is “How comfortable are you now using PAP therapy on a scale of 1-10 with 10 equal to super comfortable?” (block 631), which is followed by the user selecting their new comfort rating (block 632). An increase of 2 or more in the comfort rating triggers the “comfortability improved” event to be identified (block 640).

The next step in this behavior protocol is at the end of the practice session when the user either sufficiently improved his or her comfort rating or the session timed out, which clears the “Daytime practice session” event (block 650). Clearing of this event triggers the protocol to notify the user the practice session has ended. An example prompt is “Way to go completing your practice!” (block 651). The session ending also triggers the protocol to schedule a nighttime reminder to use PAP therapy at the user's preferred time (block 660). An example prompt is “Let's keep the momentum going and schedule a reminder to use your PAP therapy tonight.” (block 661), which is followed by the user selecting their preferred reminder time (block 662).

Clearing of the “Daytime practice session” event (block 650) and scheduling the “PAP use reminder” alarm (block 670) causes the “Daytime practice” behavior protocol to terminate (block 680).

The above example of a behavior protocol initiating and terminating a daytime practice session is only one example. Other protocols may include additional or alternative interaction with the user and/or alternative thresholds for comfort ratings and/or maximum/minimum times through the loop (block 600).

During this process, the BTS 100 continuously collects data and identifies the occurrence of other events. The training module 140 manages the coordination of the behavioral training protocols including determining how an event impacts any running behavioral training protocols as well as whether an identified event requires a new protocol to be initiated. For instance, if during the “Daytime practice” protocol the user removes the PAP mask 220, the BTS would not initiate the “Mask removed” protocol as would occur at night, but instead the practice would be paused and coordination with the user would occur to either replace the mask, re-start the practice after delivering necessary information to the use, or reschedule the daytime practice.

### Example

Minor levels of air leak can occur between the mask and the user's face that would not interrupt the PAP machine from continuing to run. However, those minor air leaks may cause discomfort for the user with a steady stream of air flow against their eyes at night. The user might not be aware of this when it occurs, but in the morning, the user may have discomfort and possibly did not obtain the maximum benefit of PAP therapy due to the air leak. The system for providing behavioral training for user engagement with medical devices can be implemented to detect the location (at the mask/user interface or at the hose/machine interface), severity (minor, medium, and high), time, and duration of air leaks. In this example, a system can include 1) two hardware modules that contain sensors and interface with the hose, mask, and machine, 2) an electronics unit with signal processing circuitry and microcontroller, and 3) a laptop running the control algorithm.

The first module containing sensors is located between the hose and the PAP mask, and the second module is located between the hose and the air output line at the PAP machine. Since the PAP hoses are flexible, the part of the module inserted into the hose is comprised of a rigid polymer while the part of the modules that needs to fit over rigid connectors on the mask and PAP machine are comprised of a flexible polymer. The majority of the module is comprised of the rigid polymer to maintain structural integrity. On the mask side, the connector can contain a single air pressure sensor, while the connector on the machine side can contain an air pressure sensor along with an audio sensor. The sensor modules can be connected to the electronics unit.

The electronics unit can comprise a single stage RC low pass filter for air pressure sensor data, a microcontroller used to interface between the filtered analog air pressure sensor data and the software program, and an audio interface that can receive an analog audio signal and output a digital signal to the software program. For example, the microcontroller can be an Arduino Uno microcontroller and the like. For example, the audio input can be a USB/audio interface and the like.

Control can be implemented in a computing environment using program instructions to the sensor data for real-time monitoring and feedback of the user's engagement with the PAP machine. In an aspect, a graphical user interface (GUI) allows users to select notification parameters prior to running the system and provides real-time updates on the sensor data and air leak events during use. The program instructions and GUI can also reflect data from a plurality of sensors, including motion sensor data to be monitored. Calibration can be performed at any time without the need for a low range reference pressure. To calibrate, both sensor modules are disconnected from the PAP hose, mask, and machine, thus exposing both pressure sensors to the same atmospheric pressure reference, then powered and monitored over a 60 second duration.

Occurrence and severity of air leak at the mask is determined through differential air pressure (P_{diff}) measurements from both air pressure sensors, as in equation (1). Pₘₐₛₖ is the pressure measured from the sensor located at the mask, and P_{machine} is the pressure measured from the sensor located at the machine. Pressure is measured in units of mmH₂O.

P_{diff}=Pₘₐₛₖ−P_{machine} (1)

The severity of each leak is based on the value of the differential pressure, for which thresholds were determined experimentally and are provided in Table 1.

TABLE 1
Experimentally determined range for the differential
pressure used to classify the severity of leak occurring
at the mask-side of the monitoring system.

| | |
|---|---|
| P_{diff} (cmH₂O) | Mask-Side Leak |

| Min. | Max. | Severity Classification |
|---|---|---|
| ≥0.7 | <1.1 | Minor |
| ≥1.1 | <3 | Medium |

| | |
|---|---|
| ≥3 | Major |

Occurrence and severity of the leaks at the machine are based on analysis of data from the audio sensor. MATLAB functions pwelch and trapz were used to obtain the Power Spectral Density (PSD) of the audio signal and then the area under the curve for the PSD. The severity of the leaks (minor, medium, and high) are based on the value of the average area under the curve over each sample (PSD_{avg_area}). Sample lengths for each analysis are 2 seconds, and thresholds in the average area relating to severity of air leaks were determined experimentally and are provided in Table 2.

TABLE 2
Experimentally determined range for the average area under the curve
of the power spectral density used to classify the severity of
leak occurring at the machine-side of the monitoring system.

| | |
|---|---|
| PSD_{avg}_{_{—}}ₐᵣₑₐ | Machine-Side Leak |

| Min. | Max. | Severity Classification |
|---|---|---|
| >6 | ≤10 | Minor |
| >10 | ≤15 | Medium |

| | |
|---|---|
| >15 | Major |

The example sensor configuration was also determined to be able to monitor respiratory rate, and thus occurrence of apnea events. Time domain analysis was performed in MATLAB first by applying a low pass filter, then performing peak detection and normalization, and finally counting of peaks. Using guidance from the clinical sleep specialist on the research team, apnea detection was based on pre-defined periods of time during which no peaks within the audio data were detected.

The control algorithm is configured to allow a user to select a notification method at the onset of a detected air leak. The methods include an alarm, email, text to a cell phone, or no notification. Once an air leak has ended, the control algorithm will again send a notification through the same pre-set method.

The user can define who the recipients are, such as their physician, caregiver, or themselves. In addition to immediate notification, the control algorithm also stores all data to a local file to be accessed for future analysis. When the program is ended by the user, a final summary report is compiled and saved locally as well.

The research team includes a clinical sleep specialist who works with individuals who have been prescribed PAP therapy and is knowledgeable of the occurrence of air leaks during PAP therapy use. This team member established a standard protocol for all team members to follow for how to manually simulate varying levels of air leak (minor, medium, and major) that realistically occur at the mask and at the machine during PAP therapy use. A data collection sheet was used for each experiment wherein the time for each event was documented as indicated by the timer running in MATLAB while the program ran. Experiments were run using a ResMed AirCurve 10 with standard flexible hose and full-face mask. Researchers ran the system with the PAP therapy running at a constant minimal pressure (4cmH₂O) applied due to safety and comfort for the user during trials. For each experiment, pre-determined breathing profiles were defined in which specific types of air leak were introduced manually for a specified duration and with set time intervals between each event. Events included starting and stopping PAP therapy, initiating and discontinuing air leaks, and occurrence of notifications. All participants involved in testing and analysis discussed in this paper were members of the research team, and Institutional Review Board approval was not required.

At the end of the experiment, the monitoring system and the PAP machine are stopped, and all the collected data is stored locally with specific information on detected leaks automatically saved to a data spreadsheet. Information from the data collection sheet is then transcribed into the data spreadsheet according to the documented times of the events. Additionally, data saved locally to the PAP machine on a memory card is extracted using an open-source EDFbrowser program. A subset of the data from the PAP machine is transcribed into the data spreadsheet for analysis purposes including machine identified time series data for mask pressure, leak, and respiratory rate. Accuracy, sensitivity, and specificity of the developed monitoring system in identifying the air leak events is then determined. Accuracy rate is the overall agreement between the simulated events and the events identified by the developed system. Sensitivity is defined as the correctly identified air leak events, calculated as the number of correctly identified simulated air leak events divided by the total number of simulated air leak events. Specificity is defined as the correctly identified non-leak events, calculated as the number of correctly identified simulated non-leak events divided by the total number of simulated non-leak events. For the calculation of each rate, events over the entire run time for each experiment were included.

Initial safety testing was conducted to determine if the monitoring technology interfered with the delivery of the prescribed PAP therapy from the PAP machine. For this test, a researcher ran the PAP system for a period of nine minutes without introducing any external events such as air leaks or changes in the breathing profile. This procedure was run both with and without the monitoring technology connected to the PAP system. Then, data from the PAP machine on “mask pressure” and “leak” were compared from both runs.

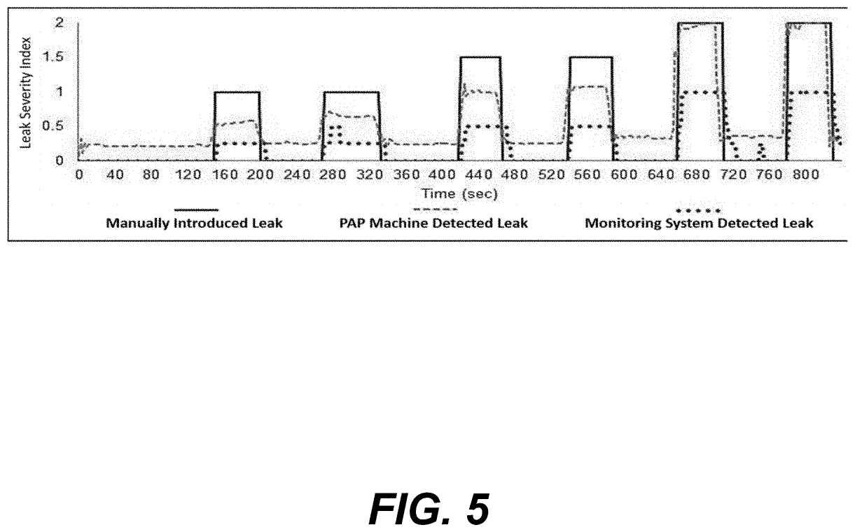

FIG. 5 shows a comparison of data from the PAP machine, the developed monitoring system, and the manually introduced events when varying levels of leak were manually introduced at the mask. Over a period of 15 minutes (900 seconds) a total of six instances of leak were manually introduced, two each at minor, medium, and major severity levels. A leak severity index is assigned as “1” for “minor”, “1.5” for “medium”, and “2” for “major”. This plot shows that the monitoring system identified the occurrence of these leak events at the same time and same relative severity as the PAP machine. Analysis showed an accuracy rate of 93.6%, sensitivity of 98.7%, and specificity of 90.9%. Discrepancies between the actual event occurrence and the response from the monitoring system is a function of the response time of the sensors and control algorithm.

To address safety of adding a monitoring system onto an existing PAP therapy system, data from the monitoring system and from the PAP machine were compared to analyze interference in the delivered PAP therapy. The pressure and leak response profiles from the PAP machine remained consistent across each run demonstrating the monitoring system did not alter or impact the delivery of the pressurized air from the PAP system. Notifications were also successfully generated and sent upon occurrence of a detected event.

Additional details of this example are provided in E. D. Clements, B. M. Roane, H. Alshabrawy, A. Gopalakrishnan and S. Balaji, “System for Monitoring User Engagement with Personalized Medical Devices to Improve Use and Health Outcomes,” 2019 41st Annual International Conference of the IEEE Engineering in Medicine and Biology Society (EMBC), Berlin, Germany, 2019, pp. 4301-4305, the entire contents of which are hereby incorporated herein by reference.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A behavioral training system, comprising:
one or more sensors configured to capture measured data, the measured data received from one or more sensors configured to measure one or more parameters pertaining to engagement of a user with a medical device;
a user interface configured to receive user input data from the user;
a computing device comprising at least one hardware processor; and
program instructions executable in the computing device that, when executed by the computing device, cause the computing device to
analyze the measured data and the user input data using an expert system,
determine, in real time, whether an event related to engagement of the user with the medical device has occurred, and
in response to the event, initiate a behavioral training protocol based on an event status.

2. The behavioral training system of claim 1, wherein the program instructions to initiate the behavioral training protocol further cause the computing device to at least one of: send information to the user to facilitate a change in engagement with the medical device and collect additional input data from the user.

3. The behavioral training system of claim 1, further comprising an electronics unit configured to collect and process measured data from the one or more sensors, wherein the measured data is processed using at least one of: digitization and amplification.

4. The behavioral training system of claim 1, wherein the one or more sensors are positioned on one or more components of the medical device, and the event status comprises identifying at least one of: a location, a severity, and a duration of the event.

5. The behavioral training system of claim 4, wherein the one or more sensors are positioned on at least one of: a device configured to generate pressurized air, a hose, and a mask.

6. The behavioral training system of claim 1, wherein the one or more sensors comprises at least one of: an air pressure sensor, an air flow sensor, an audio sensor, an inertial motion sensor, a light sensor, and a temperature sensor.

7. The behavioral training system of claim 1, further comprising wireless communication between at least one of: one or more of the sensors, electronics unit, controller, and user interface.

8. The behavioral training system of claim 1, wherein the user interface is incorporated into a smart phone, tablet, or wearable device.

9. The behavioral training system of claim 1, wherein the one or more sensors is worn by the user.

10. The behavioral training system of claim 1, wherein the program instructions further cause the computing device to terminate the behavioral training protocol in response to a change in the event status.

11. A behavioral training system in relation to positive airway pressure (PAP) therapy, the system comprising:
a computing device comprising at least one hardware processor; and
program instructions executable in the computing device that, when executed by the computing device, cause the computing device to
measure one or more parameters associated with an operation of a PAP system for PAP therapy using one or more sensors, wherein the PAP system comprises a PAP device that generates pressurized air, a hose, and a mask, wherein the hose delivers the pressurized air to the mask configured to be worn by a user;
collect information from a user of the PAP system pertaining to engagement of the user with the PAP system;
receive the measured parameters and the collected information;
analyze the parameters and the information to identify one or more behavioral training protocols designed to increase adherence of the user to the PAP therapy; and
apply the one or more behavioral training protocols.

12. The system of claim 11, wherein the one or more sensors are positioned at the interface of the mask and the user or at the interface of the hose and the PAP device.

13. The system of claim 11, wherein the one or more sensors comprises at least one of: an air pressure sensor, an air flow sensor, an audio sensor, an inertial motion sensor, a light sensor, and a temperature sensor.

14. The system of claim 11, wherein the one or more behavioral training protocols are identified based on an event, the event comprise at least one of: the PAP device turned on, the PAP device turned off, the user wearing the mask while the PAP device is on, the user has removed the mask while the PAP device is on, the PAP system not used for a period of time, the PAP system terminated before treatment period complete, and practice session.

15. The system of claim 11, wherein analyzing the parameters and the information to identify one or more behavioral training protocols comprises evaluating one or more preceding events that have occurred, time of day, user preferences, and behavior protocol requirements.

16. A method for behavioral training, comprising:
identifying parameters associated with operation of a medical device indicating engagement of a user with the medical device;
capturing measured data, the measured data received from one or more sensors configured to measure one or more parameters pertaining to engagement of the user with the medical device;
receiving user input data;
analyzing, in at least one computing device, the measured data and the user input data using an expert system;
determining, in at least one computing device, in real time, whether an event related to engagement of the user with the medical device has occurred, and
in response to the event, initiating a behavioral training protocol, in at least one computing device, based on an event status and displaying or requesting information in a user interface.

17. The method for behavioral training of claim 16, further comprising terminating the behavioral training protocol in response to a change in the event status.

18. The method of claim 16, wherein a user interface is presented to the user in a user device.

19. The method of claim 16, wherein the at least one computing device comprises a controller configured to control operation of a behavioral training system and a training module that comprises software configured to perform expert system analysis to identify and apply the one or more behavioral training protocols.

20. The method of claim 16, wherein the medical device comprises a PAP device that generates pressurized air, a hose, and a mask, wherein the hose delivers the pressurized air to the mask configured to be worn by the user, wherein the one or more sensors are positioned at the interface of the mask and the user or at the interface of the hose and the PAP device.
